(19) United States
(12) Patent Application Publication
(10) Pub. No.: US 2009/0198154 A1
(43) Pub. Date: Aug. 6, 2009

(54) Mole tracking system

(76) Inventors: Babak Roshdieh, Irvine, CA (US); Mohamed-Reza Mortazavi-Tabrizi, Santa Ana, CA (US)

Correspondence Address: RICHARD A. RYAN;ATTORNEY AT LAW, 8497 N. MILLBROOK AVENUE, SUITE 101, FRESNO, CA, 93720, US

(21) Appl. No.: 12/012,692

(22) Filed: Feb. 1, 2008

(51) Int. Cl. A61B 5/103

(52) U.S. Cl. 600/587

(57) ABSTRACT

A mole tracking system that assists with the self-examination of the human body to identify skin lesions which can be potentially cancerous. In the preferred embodiment, the system is provided as a kit having a mole measuring device, a mole documentation chart and various educational materials to help non-medical persons identify, track, record and report possibly cancerous moles in order to improve the early identification and treatment of skin cancer. The mole measuring device is a transparent sheet having a plurality of concentric circles that are spaced 1 mm apart to measure a mole and identify irregular moles. The documentation chart facilitates recording data about the mole obtained from the examination for future comparison. The educational materials tells the user how to use the kit and how to avoid skin cancer. The kit can also include a plurality of anatomical cards, a radiation detector card and sample skin protection lotion.

### CROSS-REFERENCE TO RELATED APPLICATIONS

None.

### STATEMENT REGARDING FEDERALLY SPONSORED RESEARCH

Not Applicable.

### BACKGROUND OF THE INVENTION

A. Field of the Invention

The field of the present invention relates generally to patient health care systems that are configured to facilitate examination of specific types of medical conditions. More particularly, the present invention relates to such health care systems that are directed to the detection, monitoring and reporting of skin lesions, such as moles and the like, by individuals. Even more particularly, the present invention relates to an improved mole tracking system that helps an individual detect, monitor and report any growth or change in the size, shape, color or other characteristics of moles on his or her body.

B. Background

The largest organ of the human body is the skin. It covers the exterior surface of the human body, protecting the vital organs, bones and muscle on the inside. Approximately one out of every three cancers diagnosed is a skin cancer and approximately one American dies of malignant melanoma, the most serious form of skin cancer, every hour. In fact, one out of five Americans are likely to develop some form of skin cancer in their lifetime. Despite these frightening statistics and the fact that the skin is generally visible, most people do not realize that a lesion or mole on their body is potentially cancerous. Although melanoma is the most serious form of cancer, it is almost one hundred percent curable, but only if it is identified and treated early enough. If not, melanoma can spread to other parts of the human body where it can be very difficult to treat and, in many cases, fatal. Melanoma, which is a malignant tumor that comes from the cells that produce the pigment melanin, is most commonly caused by exposure of the skin to the sun's ultraviolet radiation. Despite the increasing incidents of skin cancer and efforts by government, academic and private groups to educate people on the potential of skin cancer, most people are generally unaware of the symptoms of skin cancer and what they should be doing to help identify potential skin cancer lesions. Unlike breast self-examination, of which most women are at least generally aware of the need for and the process of performing, there is not an easily available system for a person to examine themselves on a regular or routine basis with regard to skin cancer.

One of the problems with diagnosing potential skin cancer lesions for a non-medical individual is separating potentially harmful or dangerous lesions from generally innocuous moles and the like. There are two basic forms of moles, those that are the normal small brown blemishes which most people have and typically appear early in life and atypical moles, which are also known as dysplastic nevi. Typically, the more moles a person has the greater his or her risk for melanoma. If properly heeded, various factors can warn a person that what he or she thinks is a normal, non-cancerous mole is actually a blemish that is or has the potential to be cancerous. These factors include asymmetry or odd shaped moles where one half is unlike the other half, moles that have scalloped or poorly defined borders, variations in the color from one area of the mole to another, moles that are larger than approximately 6 mm and moles that have evolved by changing color, size, shape or elevation. While not necessarily definitive of a cancerous lesion, these factors are all indicators that a person should be aware of with regard to lesions on their body. Naturally, when a person identifies moles that have these characteristics, he or she must immediately contact their doctor or a certified dermatologist to have the lesion checked and, if necessary, removed or otherwise treated.

As stated above, one of the major problems with the prevention and treatment of skin cancer is that people do not generally recognize that they have a mole or lesion that needs to be checked to determine if it is melanoma or one of the other forms of skin cancer. One reason for this problem is that there is no convenient, easy to utilize testing method to raise a person's awareness as to a possible cancerous lesion on their body. Most people simply are not familiar with the characteristics that suggest a potentially cancerous mole, are not aware of the various moles and lesions on their body and do not have a way of noticing whether a mole has appeared or changed over time in a manner that requires evaluation by a doctor. As such, people often do not make an appointment with a dermatologist to review a mole until the growth of the lesion has advanced, by which time it may require more extensive and/or aggressive medical treatment or surgery to remove the lesion. Unfortunately, sometimes it is simply too late to take medical action to avoid the cancerous growth being fatal. Having the ability to identify, track and report moles would benefit many people by helping them more quickly identify potentially cancerous lesions.

Presently, the most common manner in which moles are identified as being potentially dangerous is for a doctor or other trained medical person to visually see the mole during an otherwise routine medical examination (i.e., one that was not for the purpose of looking for moles). There are pamphlets, books, notices and other written or otherwise published materials that can assist a person with identifying moles that should be reviewed by a doctor. Various machines and other devices, some of which are very expensive, can help identify moles as being potentially cancerous. In addition, there are computer tracking systems that can be utilized to identify these lesions. Many of the current mole tracking systems are either too complicated for the average person to use, not convenient for regular use and/or are inadequate with regard to the ability to assist the user with tracking moles.

Several prior art tracking systems are the subject of issued patents. For instance, U.S. Pat. No. 6,692,032 to Christy describes a mole monitoring system comprising a record keeping system having a flexible sheet with multiple transparent regions that function as mole tracing areas for the user to place over a mole to trace an outline of the mole onto the sheet. The system uses an alphanumeric coding system to allow the user to record and track various characteristics, such as color and elevation. The sheet allows entry of readings over time so the user can track any change in the mole, record those changes and, if changes are noted, contact a health care professional. A thin-tipped marking pen is provided for the tracing. One limitation of this system is the ability of the person to accurately trace the mole from one time to another, particularly in light of the inherent inaccuracies of the width of the pen tip and the potentially very small changes in the mole. Another limitation with regard to this system is that the tracing of the mole does not allow a person to effectively communicate with a doctor or other medical person over the telephone, Internet or the like the relative change in size of the mole, particularly due to the fact that very small changes, such as 1 mm to 2 mm, can be very significant for a mole.

U.S. Pat. No. 5,018,531 to Harman, discloses a skin blemish measuring and recording system comprising a measuring device having a color reference scale for determining the color and shade of color of the blemish, a size measuring scale for measuring the size of the blemish and a distance scale for measuring the location of the mole relative to preset reference points on a body map. The data can be recorded on a flat map or chart of the human body or be entered into a computer database using a computer program having a map or chart of the human body. U.S. Pat. No. 6,427,022 to Craine, et al. discloses an image comparator system and method for detecting changes in skin lesions that utilizes a digital image of the skin, directly recorded or from a photograph, to create a stored baseline image that is compared to later images to visually identify new or growing lesions. While these two patents generally describe mole tracking systems that can be utilized to detect new moles and/or changes in existing moles, neither system is very easy for the typical individual to perform at home and to communicate to a doctor or other medical person the results of his or her examination.

What is needed, therefore, is an improved mole tracking system that better enables a person to examine himself or herself for new moles and for any changes in existing moles. The desired mole tracking system should be easy for a non-medical person to identify, track and record any new or changed moles. The preferred mole tracking system will also facilitate the person utilizing the system to report his or her findings to a medical specialist in a manner that more easily enables the medical specialist to know whether to be concerned or not. A mole tracking system will preferably include devices for measuring and recording any new or changed moles, information on how to identify moles and other skin lesions, information on how to prevent skin cancer and lotion or other materials to assist the user with preventing skin cancer.

### SUMMARY OF THE INVENTION

The improved mole tracking system of the present invention solves the problems and provides the benefits identified above. That is to say, the present invention discloses an improved mole tracking system that is designed for the general consumer to more efficiently and effectively examine themselves in order to identify, track and record new and/or changed moles or other skin lesions. The mole tracking system of the present invention is very easy for the non-medical person to utilize and allows the person to record the information in a manner that is more easily reported to a doctor or other medical person over the telephone, Internet or by other communication means. Instead of depending on the inherent variances that are susceptible with the tracing of moles or other skin lesions, the present mole tracking system allows the non-medical consumer to more specifically identify any change in size, shape, color, elevation and other characteristics that could warn that the lesion is potentially cancerous. In its preferred form, the mole tracking system of the present invention is contained in a kit having information regarding the identification, tracking and recording of new or changed moles, information on how to prevent skin cancer, devices for measuring and recording moles on the user's body and lotion that can be utilized to assist in preventing skin cancer.

As set forth herein, the improved mole tracking system of the present invention is provided in an easy-to-use kit that can assist persons with the early identification, effective tracking and useful recording of moles or other skin lesions so that the examination data may be easily reported to his or her doctor in order to improve the early detection of potentially cancerous skin lesions. The present mole tracking system allows non-medical consumers to more effectively and efficiently check for new or changed moles in the privacy of his or her own home and then report the findings to a doctor or other medical person so that it can be determined if further action is necessary. The mole tracking system of the present invention can be provided in a relatively inexpensive kit that is generally affordable to most consumers.

In a primary embodiment of the present invention, the mole tracking system comprises a mole measuring device, a mole documentation chart and one or more educational materials. The preferred mole measuring device comprises a transparent sheet that has a plurality of measurement indicators thereon for measuring a mole by being placed over the mole. In a preferred embodiment, the measurement indicators comprise a plurality of concentric circles that emanate from a center point and are spaced apart 1 mm. In another embodiment, the measurement indicators comprises an x-y scale having a plurality of distance markers that are spaced apart 1 mm. The preferred mole documentation chart is configured to assist the user with recording the size, location and color of the mole. Preferably, the chart has a plurality of rows, with one row being numbered for each mole, and a plurality of columns to record the data for the mole. The preferred chart has a column for including a rendering of the mole, such as a photograph or drawing. The educational materials explain use of the system to track, record and report moles and provide information to help the user prevent skin cancer. The educational materials can include an audio/visual disk, such as a CD or DVD, an educational pamphlet and/or an information card. Preferably, these materials include photographs or highly detailed drawings to show the user the various aspects of normal and potentially cancerous moles. The preferred mole tracking system also includes a radiation detector card that can be placed in the sun to indicate the radiation level of the sun's rays, one or more anatomical cards that depict various views of the human body for recording the location of the moles found through self-examination and one or more samples of lotion that can be utilized to reduce the likelihood of damage to the skin when exposed to the sun. Preferably, the above components are disposed within a container, such as a box or the like, and provided to the user as a kit. Use of the present mole tracking system will help the user prevent skin cancer and more quickly identify potentially cancerous skin lesions so he or she may receive early and, therefore, more effective treatment.

Accordingly, one of the primary aspects of the present invention is to provide a mole tracking system that provides the advantages discussed above and overcomes the disadvantages and limitations associated with presently available systems and methods of identifying, recording and reporting new moles and/or changes in existing moles or other skin lesions.

It is also an important aspect of the present invention to provide a mole tracking system that is easy for the non-medical consumer to utilize so that he or she may better identify, track and record new moles or moles which have changed so as to improve the likelihood of early identification of possibly cancerous lesions.

It is also an important aspect of the present invention to provide a non-technical mole tracking system that is useful for helping the typical consumer improve the likelihood that possibly cancerous lesions on their body will be identified in the early stages so as to provide for prompt and effective medical treatment.

It is also an important aspect of the present invention to provide a mole tracking system that reduces the inaccuracies inherent in self-examination procedures by providing the consumer with the ability to better identify new and changed skin lesions and to present such information to a doctor or other medical person so that the information may be evaluated for potential further action.

It is also an important aspect of the present invention to provide a mole tracking system that encourages non-medical persons to take a more active role in identifying potentially cancerous lesions on their body or that of their loved ones and in preventing the occurrence of conditions that can lead to or cause skin cancer.

Other aspects and advantages of the mole tracking system of the present invention will be readily apparent to those skilled in the art in light of the figures and description set forth herein.

The above and other aspects of the present invention are explained in greater detail by reference to the attached figures and the description of the preferred embodiment which follows. As set forth herein, the present invention resides in the novel features of form, construction, mode of operation and combination of processes presently described and understood by the claims.

### BRIEF DESCRIPTION OF THE DRAWINGS

In the drawings which illustrate the preferred embodiments and the best modes presently contemplated for carrying out the present invention:

FIG. 1 is a top view of the components of a kit configured according to a preferred embodiment of the mole tracking system of the present invention;

FIG. 2 is a top plan view of the embodiment of the mole measuring device of the kit shown in FIG. 1 having concentric circles;

FIG. 3 is a top plan view of an alternative embodiment of the mole measuring device for the kit of FIG. 1 having an x-y scale; and

FIG. 4 is a top plan view of the mole documentation sheet of the kit of FIG. 1.

### DETAILED DESCRIPTION OF THE PREFERRED EMBODIMENTS

With reference to the figures where like elements have been given like numerical designations to facilitate the reader's understanding of the present invention, the preferred embodiments of the present invention are set forth below. The enclosed figures and drawings are merely illustrative of one or more of the preferred embodiments and, as such, represent one or more ways of configuring the present invention. Although specific components, materials, configurations and uses are illustrated, it should be understood that a number of variations to the components and to the configuration of those components described herein and in the accompanying figures can be made without changing the scope and function of the invention set forth herein. For instance, although the figures and description provided herein are primarily directed to a mole tracking system for use in the self-examination and tracking of potentially cancerous skin lesions, those skilled in the art will readily understand that this is set forth merely for purposes of simplifying the present disclosure and that the present invention is not so limited. For instance, the mole tracking system of the present invention can be utilized by persons, including medical personnel, to identify, record and report skin lesions of another.

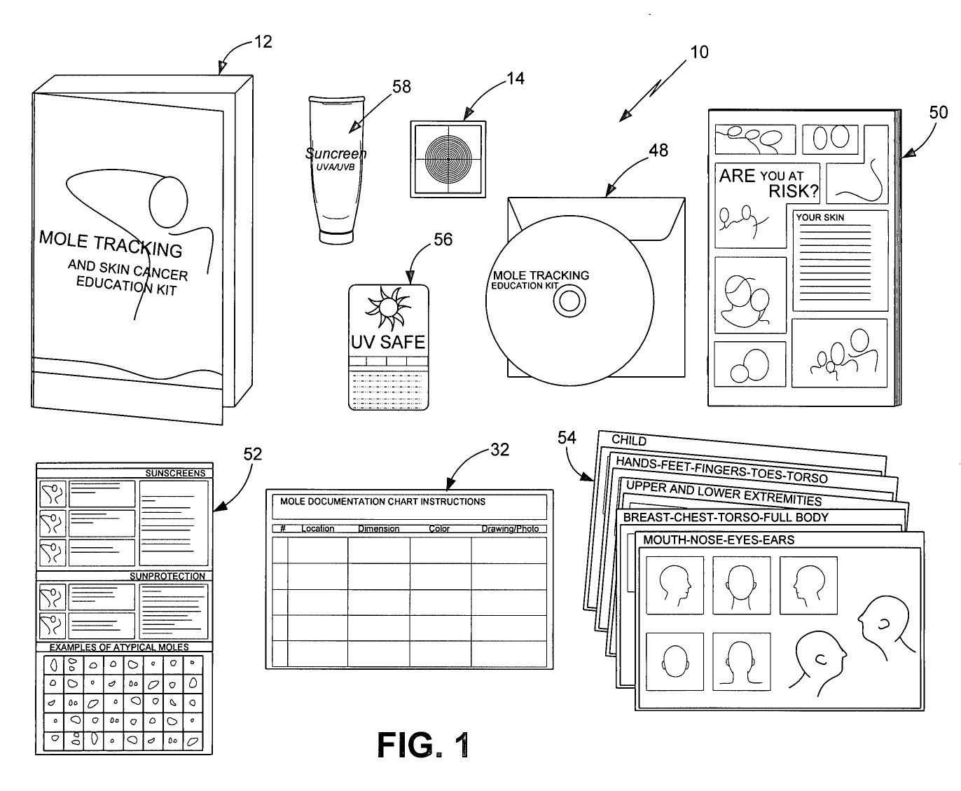

A mole tracking kit that is manufactured out of the components and configured pursuant to a preferred embodiment of the mole tracking system of the present invention is shown generally as 10 in FIG. 1. The mole tracking kit 10 of the present invention is preferably and beneficially utilized to help non-medical persons combat skin cancer through the early detection, by self-examination, and reporting of possibly cancerous moles or other skin lesions and by educating persons with regard to the causes and prevention of skin cancer. The preferred embodiment of the mole tracking kit 10 of the present system comprises various components that are specially configured to allow a non-medically trained person to examine himself or herself so as to identify new moles and/or any moles which have changed over time. In addition, the mole tracking kit 10 has components to simplify the tracking of moles and the reporting of any changed conditions to a doctor or other medical person. Unlike prior art mole tracking systems, the kit 10 of the present mole tracking system more specifically identifies small changes in the size and/or condition of a mole so that the user may more quickly identify that the mole has changed, report those changes to a doctor and obtain early medical treatment to remove or otherwise treat the mole.

As shown in FIG. 1, the mole tracking kit 10 of the system of the present invention preferably comprises a container 12 configured to hold the various other components, set forth below, of the present mole tracking system. In the embodiment shown, container 12 is a cardboard box that is specially configured to receive and hold the other components. As well known to those skilled in the art, however, container 12 can be made out of a wide variety of different materials and configured in a wide variety of different shapes. For instance, container 12 can be made out of plastic and configured as a clam-shell or shrink-wrap type of package. Preferably, the container 12 is sized to neatly and in an organized manner hold the various components of the system of the present invention so these components are not loosely contained therein, which can result in damage to the components, and so they can be readily and easily accessible by the user of mole tracking kit 10 when in use.

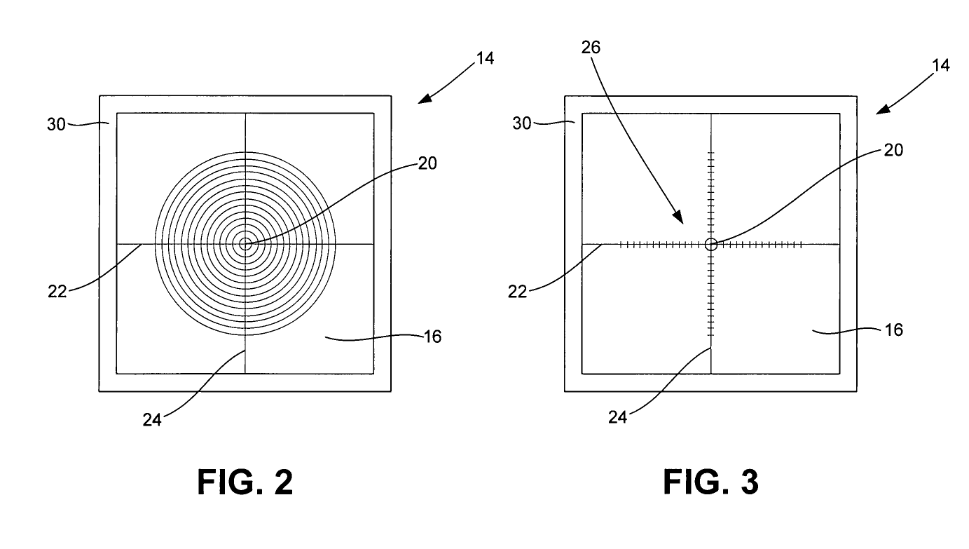

One of the primary components of the mole tracking system of the present invention, and therefore part of mole tracking kit 10, is a mole measuring device 14 that is utilized to measure the size and dimension of a mole. The mole measuring device 14 of the present invention comprises a transparent sheet 16 having a plurality of measurement indicators thereon. In the embodiment of FIG. 2, the measurement indicators are a plurality of concentric circles 18 that radiate outward from the center point 20 defined by the intersection of perpendicularly disposed x-y lines, shown as 22 and 24, respectively. Preferably, the concentric circles 18 are each equally spaced apart a distance that is easy for the user to distinguish when he or she is utilizing the mole measuring device 14 to measure the size of a mole. In the preferred embodiment, the concentric circles 18 are each spaced apart 1 mm, such that each concentric circle 18 represents an additional 1 mm in radial length from the center point 20 along the x-y lines 22/24. In use, the user of mole tracking kit 10 of the present system places the mole measuring device 14 over the mole, with the center point 20 at the center of the mole to measure the size of the mole. Because the device 14 has transparent sheet 16, the user will be able to see the mole through the device 14. A symmetrical mole will be easily seen as falling within one of the concentric circles 18 on mole measuring device 14. As will be easily noticed by the user, an asymmetrical or irregular mole will have edges that extend beyond the rounded, perfect circle of one of the concentric circles 18. Noticing and recording the irregularities of a mole is very important to detecting potentially cancerous moles. The use of the concentric circles 18 on mole measuring device 14 provides the user with a visually easy means of identifying moles which have asymmetrical or irregular edges. The user also utilizes the concentric circles 18 to measure the irregular shaped mole.

In the alternative embodiment of the mole measuring device 14 shown in FIG. 3, the device 14 comprises an x-y scale 26 having the x-y lines 22/24 each with a plurality of spaced apart distance markers 28 thereon. As with the concentric circles 18, the distance markers 28 extend outwardly from the center point 20 and are preferably equally spaced apart a distance that is easily distinguished by the user. In the preferred embodiment, the distance markers 28 are each spaced apart 1 mm, beginning at the 0,0 or center point 20. The user utilizes the x-y scale 26 to measure the “x” and “y” dimensions of the mole. If desired, the transparent sheet 16 of mole measuring device 14 can include both concentric circles 18 and the x-y scale 26 and be provided with a border 30.

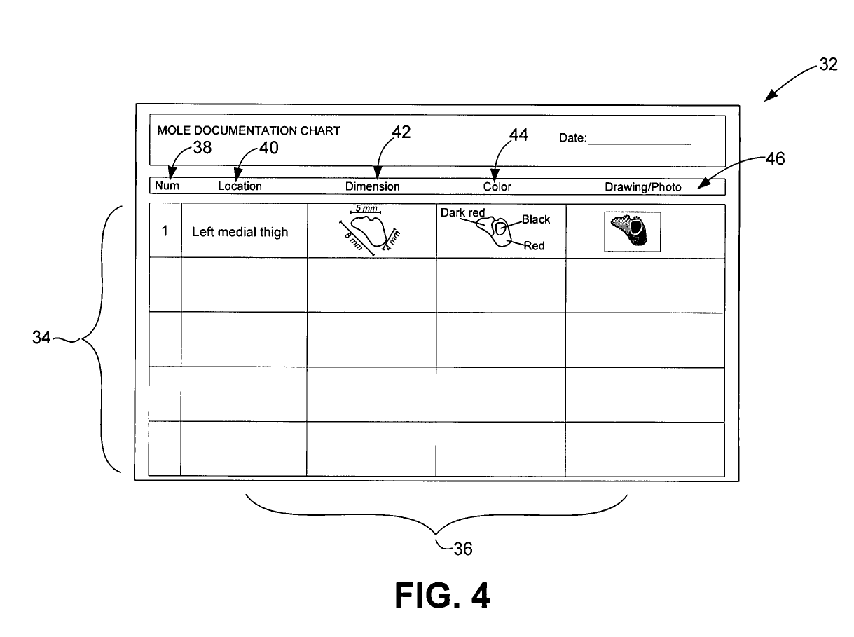

The mole tracking kit 10 of the system of the present invention also includes a mole documentation chart 32, best shown in FIG. 4, for recording the size, location and color of the mole. In the preferred embodiment, the mole documentation chart 32 comprises a plurality of rows 34 and a plurality of columns 36. The plurality of rows 34 are typically utilized for different moles and are numbered consecutively beginning with the number 1, as shown, in the first column 38. The number assigned to each row is also utilized to document the position of the mole on the anatomical charts described below. The remaining plurality of columns 36 are used to describe details about the various moles. As shown in FIG. 4, a second column 40 is utilized to describe the location of the mole on the human body, such as “left medial thigh” or other locations. As set forth below, anatomical information is also included in mole tracking kit 10 to assist the user with identification of the location of the mole. A third column 42 is utilized to describe the dimension or size of the mole. As will be readily understood by those skilled in the art, while a symmetrical mole can be identified by its radius or diameter, an asymmetrical mole may be best described by utilizing various side dimensions (as shown). For asymmetrical moles, it may also be useful to include a small drawing of the mole to better indicate the size of the mole. Fourth column 44 is utilized to indicate the overall color of the mole. If the mole is multicolored, it may also be useful to include a drawing of the mole to indicate the various colors of the mole and where the colors are located on the mole. As known to those skilled in the art, moles may have colors such as white, yellow, tan, light brown, brown, dark brown, black and red. While it is important to accurately describe the color or colors of the mole, it is even more important that the user be consistent in describing the color(s) of moles so that changes in the color of a mole can be easily and quickly noted. The fifth column 46 is utilized to place a rendering of the mole, such as a close-up digital photograph of the mole or a drawing that shows the mole, on the mole documentation chart for future comparison. Naturally, it is best that the rendering be as detailed and accurate as possible so that the user can easily spot even small changes in the mole during future examinations. With its record keeping function, the mole documentation chart 32 is the heart of the mole tracking kit 10 of the system of the present invention.

Also included in mole tracking kit 10 of the present invention are one or more educational materials that explain use of the system and mole tracking kit 10 of the present invention to track, record and report moles and to provide information to the user that will assist him or her to prevent skin cancer. In a preferred embodiment, the mole tracking kit 10 comprises an audio/visual disk 48, typically in the form of a CD and/or DVD, that can be played on a computer, DVD player or other device. The audio/visual disk 48 can contain instructions, hints, suggestions, advice and background on utilizing the mole tracking kit 10 of the present invention. As well known to those skilled in the art, audio/visual disk 48 can contain written text, drawings, photographs and video sections that show the user how to use the mole measuring device 14 to measure the mole, how to use the mole documentation chart 32 to record and track the progress of the moles and how to use the other components of mole tracking kit 10. In addition to instructions on the system, the audio/visual disk 48 can contain information regarding the dangers of over-exposure to the sun, the growth of cancerous moles, various treatment options for treating moles that are cancerous and advice on how to avoid skin cancer and other skin-related problems. The mole tracking kit 10 of the present invention also includes an educational pamphlet 50 that contains much of the same information as the audio/visual disk 48, although typically in somewhat less detail due to the medium. Preferably, both the educational pamphlet 50 and the audio/visual disk 48 include a glossary of anatomical terminology that will assist the lay person with understanding the various terms which are utilized to describe moles, skin cancer and certain components of the mole tracking kit 10. The educational materials of the preferred embodiment of the mole tracking kit 10 also includes a “quick facts” card 52 that includes summarized information pertaining to the identification of moles, use of sunscreens and/or other lotions to prevent skin cancer and use of the mole tracking kit 10. To assist the user, the audio/visual disk 48, educational pamphlet 50 and/or the quick facts card 52 preferably should include clear, detailed photographs of various regular and irregular moles to provide the user with a useful level of knowledge regarding the differences between normal and potentially dangerous moles. In addition to the above, the educational materials of the mole tracking kit 10 of the present system can include a variety of other types of text, drawings, photographs or the like, including other types of video and/or audio materials.

The preferred embodiment of the mole tracking kit 10 of the system of the present invention also includes one or more anatomical cards 54 that have drawings and/or photographs depicting the human body. The anatomical cards 54 are utilized to document the position of the moles that are listed on the mole documentation chart 32. As set forth above, each mole is given a number that is recorded in the first column 38. That same number is marked on an anatomical card 54 at the position where the mole is located on the body. In a preferred embodiment, the mole tracking kit 10 has a plurality of anatomical cards 54 that each depicts a certain region of the body so as to enable the user to more easily position the appropriate mole number on the anatomical cards 54. For instance, one card can be utilized to show various angles of the human head to allow the user to mark moles on or near the mouth, nose, eyes and ears. Preferably, the user will utilize lines and/or arrows to demonstrate the correct angle and distance from various body landmarks, such as the knee, elbow, wrist or the like. Utilizing the anatomical cards 54, the user can more easily determine when a new mole has appeared and identify those moles that have changed over time, both being critical to the prevention of skin cancer.

Also included with the mole tracking kit 10 of the present invention is a radiation detector card 56. In one embodiment, the radiation detector card 56 includes a UVB sensor which can be utilized to determine the amount of UVB exposure that a person will be exposed to so that he or she can take appropriate action to reduce the likelihood of harm from such exposure. Preferably, radiation detector card 56 is sized to be about the same dimensions as a credit card so that it can fit into the user's pocket or wallet, thereby allowing the user to carry it with him or her for use as needed. One preferred type of radiation detector card 56 requires the UVB sensor be placed under direct sunlight for a certain period of time, such as twenty seconds, to allow the sensor to change color. The change in color can be compared to a color scale to access the amount of UVB exposure that the individual will be receiving. Various different types of radiation detector cards 56, many being generally commercially available, can be utilized with the mole tracking kit 10 of the system of the present invention.

In a preferred embodiment, the mole tracking kit 10 of the system of the present invention also includes one or more containers, such as tubes, of lotion 58 that can be utilized by the user to prevent skin damage that can lead to moles and/or skin cancer. The lotion should be configured to protect human skin from the damaging effects of the sun's rays. Preferably, the lotion 58 included with the mole tracking kit 10 is of a type that is much more effective for protecting the skin than the sunscreen lotions which are commercially available, most of which contain various ingredients that are only included in the lotion for cosmetic or other non-medical reasons and which are generally useless or of very little value for protecting the skin. The lotion 58 included with the mole tracking kit 10 can be custom made for use therewith, can be an available lotion or can be purchased from manufacturers who make such lotions.

In addition to the foregoing components, various other items can be included with the mole tracking kit 10 of the system of the present invention as desired to assist the user with identifying, tracking and recording moles on his or her body and with preventing skin problems that can lead to skin cancer. In use, the user opens the container 12 and removes the various components therefrom. After he or she has read the instructions and reviewed the educational materials, then the user will perform a self-examination to locate the moles on his or her body. The user then utilizes the mole measuring device 14 to measure the size of each mole and records that information on the mole documentation chart 32. As set forth above, the user also includes the location, size and color of each mole on the chart 32 and includes a rendering, typically either a photograph or a drawing, in the fifth column 46 on the chart 32. Preferably, the user will perform a self-examination at least monthly to keep up to date with any new moles and to quickly identify any changes in a mole that may indicate that the mole is or may be cancerous.

As set forth above, the mole tracking kit 10 of the system of the present invention is utilized to help the consumer combat skin cancer through self-examination in order to substantially improve the likelihood of early detection and reporting of possibly cancerous moles or other skin lesions and by educating such persons with regard to the causes and prevention of skin cancer. The preferred embodiment of the mole tracking kit 10 comprises components which are specially selected and configured to allow a non-medically trained person to effectively exam himself or herself in a manner that facilitates identifying new moles and/or any changes in existing moles. The mole tracking kit 10 has components that simplify the locating and tracking of moles and the reporting of any changed conditions to a doctor or other medical person. As such, the mole tracking kit 10 of the present system will reduce the likelihood that a person will require sophisticated surgery or other advanced medical treatment or die due to skin cancer.

While there are shown and described herein specific forms of the invention, it will be readily apparent to those skilled in the art that the invention is not so limited, but is susceptible to various modifications and rearrangements in design and materials without departing from the spirit and scope of the invention. In particular, it should be noted that the present invention is subject to various modifications with regard to any dimensional relationships set forth herein and modifications in assembly, materials, size, shape and use. For instance, there are numerous components described herein that can be replaced with equivalent functioning components to accomplish the objectives of the present invention.

What is claimed is:

1. A mole tracking system, comprising:
a mole measuring device comprising a transparent sheet having a plurality of measurement indicators thereon for measuring a mole by being placed over the mole;
a mole documentation chart for recording the size, location and color of the mole; and
one or more educational materials explaining use of said system to track, record and report moles and providing information to prevent skin cancer.

2. The mole tracking system according to claim 1, wherein said measurement indicators are configured as a plurality of concentric circles.

3. The mole tracking system according to claim 1, wherein said measurement indicators are configured as an x-y scale having a plurality of spaced apart distance markers.

4. The mole tracking system according to claim 3, wherein said measurement indicators further comprise a plurality of concentric circles.

5. The mole tracking system according to claim 4, wherein each of said distance markers and each of said concentric circles are spaced apart a distance of 1 mm.

6. The mole tracking system according to claim 1, wherein said mole documentation chart further comprises a plurality of data rows and a plurality of data columns configured to receive mole data, said data columns including a rendering column for receiving a rendering of the mole.

7. The mole tracking system according to claim 1, wherein said educational materials include an audio/visual disk.

8. The mole tracking system according to claim 7, wherein said educational materials include an educational pamphlet.

9. The mole tracking system according to claim 1, wherein said educational materials comprise at least one of an audio/visual disk and an educational pamphlet.

10. The mole tracking system according to claim 1 further comprising a radiation detector card.

11. The mole tracking system according to claim 1 further comprising one or more anatomical cards depicting the human body.

12. The mole tracking system according to claim 1 further comprising a lotion to prevent skin cancer.

13. The mole tracking system according to claim 1 further comprising at least one of a radiation detector card, one or more anatomical cards and a lotion to prevent skin cancer.

14. A mole tracking system, comprising:
a mole measuring device comprising a transparent sheet having a plurality of measurement indicators thereon for measuring a mole by being placed over the mole, said measurement indicators comprising either a plurality of concentric circles or an x-y scale having a plurality of spaced apart distance markers;
a mole documentation chart having a plurality of data rows and a plurality of data columns configured to receive mole data, said data columns including columns for recording the size, location, color and a rendering of the mole; and
one or more educational materials explaining use of said system to track, record and report moles and providing information to prevent skin cancer, said educational materials comprising at least one of an audio/visual disk and an educational pamphlet.

15. The mole tracking system according to claim 14, wherein said concentric circles and said distance markers are spaced apart a distance of 1 mm.

16. The mole tracking system according to claim 14 further comprising at least one of a radiation detector card, one or more anatomical cards and a lotion to prevent skin cancer.

17. A mole tracking and cancer prevention kit, comprising:
a mole measuring device comprising a transparent sheet having a plurality of measurement indicators thereon for measuring a mole by being placed over the mole, said measurement indicators comprising either a plurality of concentric circles or an x-y scale having a plurality of spaced apart distance markers;
a mole documentation chart having a plurality of data rows and a plurality of data columns configured to receive mole data, said data columns including columns for recording the size, location, color and a rendering of the mole;
one or more anatomical cards depicting various parts of the human body; and
one or more educational materials explaining use of said system to track, record and report moles and providing information to prevent skin cancer, said educational materials comprising at least one of an audio/visual disk and an educational pamphlet.

18. The mole tracking system according to claim 17, wherein each of said distance markers and each of said concentric circles are spaced apart a distance of 1 mm.

19. The mole tracking system according to claim 17 further comprising at least one of a radiation detector card, one or more anatomical cards and a lotion to prevent skin cancer.

20. The mole tracking system according to claim 17, wherein each of said mole measuring device, said mole documentation chart, said anatomical cards and said educational materials are disposed within a container for said mole tracking kit.
